(19) United States
(12) Patent Application Publication
(10) Pub. No.: US 2022/0087696 A1
(43) Pub. Date: Mar. 24, 2022

(54) PERCUTANEOUS RETROGRADE THERAPY FOR PULMONARY EMBOLISM

(71) Applicant: EAST END MEDICAL, LLC, Lewes, DE (US)

(72) Inventors: BRIJESHWAR S. MAINI, West Palm Beach, FL (US); Saif Anwaruddin, Shrewsbury, MA (US)

(21) Appl. No.: 17/478,498

(22) Filed: Sep. 17, 2021

Related U.S. Application Data
(60) Provisional application No. 63/080,332, filed on Sep. 18, 2020.

(51) Int. Cl. A61B 17/221

(52) U.S. Cl. CPC A61B 17/221; A61B 2017/22079; A61B 2017/2212

(57) ABSTRACT

The disclosed invention provides a closed loop catheter system for treatment of pulmonary embolism with balloon tipped catheter insertion devices. The system includes a first catheter insertion device configured to be positioned at proximal segment of a designated pulmonary vein and a second catheter insertion device configured to be positioned at proximal segment of a designated pulmonary artery. The first and second catheter insertion devices each include a sheath defining at least one lumen therein and at least one balloon that is connected to the distal end of the sheath. The at least one balloon seals ostium of the pulmonary vein or ostium of the pulmonary artery when inflated and the first catheter insertion device is in use.

### CROSS REFERENCE TO RELATED APPLICATIONS

This application claims the priority of U.S. Provisional Application Ser. No. 63/080,332, filed on Sep. 18, 2020, which is hereby incorporated herein by reference in its entirety.

### FIELD

The present invention relates generally to cardiac catheter system and method with balloon tipped catheter insertion devices which are suitable for facilitating precise and safe cannulation of pulmonary veins or pulmonary arteries to provide treatment for pulmonary embolism.

### BACKGROUND

Cardiac catheterization is a medical procedure in which a long thin tube or catheter is inserted through an artery or vein into specific areas of the heart for diagnostic or therapeutic purposes. More specifically, cardiac chambers, vessels and valves may be catheterized.

Cardiac catheterization may be used in procedures such as coronary angiography and left ventricular angiography. Coronary angiography facilitates visualization of the coronary vessels and finding of potential blockages by taking X-ray images of a patient who has received a dye (contrast material) injection into a catheter previously injected in an artery. Left ventricular angiography enables examination of the left-sided heart chambers and the function of the left sided valves of the heart, and may be combined with coronary angiography. Cardiac catheterization can also be used to measure pressures throughout the four chambers of the heart and evaluate pressure differences across the major heart valves. In further applications, cardiac catheterization can be used to estimate the cardiac output, or volume of blood pumped by the heart per minute.

Some medical procedures may require catheterization into the left atrium of the heart. For this purpose, to avoid having to place a catheter in the aorta, access to the left atrium is generally achieved by accessing the right atrium, puncturing the interatrial septum between the left and right atria of the heart, and threading the catheter through the septum and into the left atrium. Transseptal puncture must be carried out with extreme precision, as accidental puncturing of surrounding tissue may cause very serious damage to the heart. In addition, transseptal puncture may require complicated instruments which are not helpful in guaranteeing the precision of the puncture.

The use of devices available today present many challenges for doctors attempting to puncture the interatrial septum and perform cardiac catheterization. Locating the interatrial septum, properly placing the distal end of the puncturing device at the desired location of the septum, safely puncturing the interatrial septum, avoiding accidental punctures, and tracking and maneuvering the catheter post-puncture, are among the many challenges facing those performing cardiac catheterization today.

Furthermore, the use of different types of catheters to treat pulmonary embolism in the cardiopulmonary circulation has emerged over the past several years. The advantage of catheter based systems to treat pulmonary embolism is to provide less invasive methods for clot removal. As such, the development of novel catheters to effectively remove clot in the setting of pulmonary embolism can be life-saving and preserve quality of life to patients. The currently available devices in the space, while effective, currently have limited utility for complete clot removal in most patients.

### SUMMARY

In order to overcome the disadvantages of the conventional art, the disclosed invention provides a closed loop catheter system including multiple catheter insertion devices for treating pulmonary embolism. The closed loop catheter system, while in use, creates an isolated segment of closed segments of the pulmonary vein, corresponding pulmonary capillaries and corresponding pulmonary artery. Pulmonary embolectomy is more efficiently performed with the isolated segment created by the closed loop catheter system of the disclosed invention. Further, by using this closed loop system, clot in the pulmonary circulation can be effectively removed by pushing it out from the pulmonary vein into a catheter placed in the corresponding pulmonary artery. To access the pulmonary vein system, transseptal access will be required as described above.

These advantages may be achieved by a catheter system for pulmonary embolism with balloon tipped catheter insertion devices. The catheter system includes a first catheter insertion device configured to be suitable for facilitating precise and safe cannulation of pulmonary veins and a second catheter insertion device configured to be suitable for facilitating precise and safe cannulation of pulmonary arteries. The first catheter insertion device includes a sheath that defines at least one lumen therein and has a distal end that is configured to be positioned at proximal segment of a designated pulmonary vein and a proximal end that is external to the patient and at least one balloon that is positioned at the distal end of the sheath. The at least one balloon is configured to seal ostium of the designated pulmonary vein when inflated. The first catheter insertion device is configured to be connected to an infuser to supply fluid to the pulmonary veins. The second catheter insertion device includes a sheath that defines at least one lumen therein and has a distal end that is configured to be positioned at proximal segment of a designated pulmonary artery and a proximal end that is external to the patient and at least one balloon that is positioned at the distal end of the sheath. The at least one balloon is configured to seal the designated pulmonary artery when inflated. The second catheter insertion device is configured to be connected to an aspirator to aspirate materials from the pulmonary arteries.

The first and second catheter insertion devices each may include a dilator movably positioned in the at least one lumen. The dilator is configured to puncture septum. The first and second catheter insertion devices each may include one or more additional lumens defined by the sheath to deliver additional fluids into the designated pulmonary vein or for flushing or aspirating. The first and second catheter insertion devices each may have a plurality of curls and flexion points for multidirectional deflections. The at least one lumen of the first catheter insertion device may be configured to deliver the fluid supplied by the infuser. The at least one lumen of the second catheter insertion device may be configured to carry the materials to the aspirator.

These advantages may be also achieved by a method of using a catheter system including a first catheter insertion device and a second catheter insertion device for pulmonary embolism. The method includes steps of engaging a distal portion of a first catheter insertion device with an ostium and proximal segment of a designated pulmonary vein, inflating at least one balloon of the first catheter insertion device to seal the ostium of the designated pulmonary vein, positioning a distal portion of the second catheter insertion device within a proximal segment of a corresponding designated pulmonary artery, inflating at least one balloon of the second catheter insertion device to seal the designated pulmonary artery, performing infusion of fluid into the designated pulmonary vein through at least one lumen defined in a sheath of the first catheter insertion device by using an infuser connected to the first catheter insertion device, and performing aspiration of materials from the designated pulmonary artery through at least one lumen defined in a sheath of the second catheter insertion device by using an aspirator connected to the second catheter insertion device.

The engaging the distal portion of the first catheter insertion device may be performed by using a fluoroscopic or echocardiographic guidance. The infusion of fluid may be performed such that the fluid flows into the designated pulmonary vein, corresponding pulmonary capillaries and the corresponding designated pulmonary artery. The infusion of fluid may be performed with a designated rate of flow and pressure to dislodge thrombotic materials from a pulmonary arterial vascular bed into the proximal segment of the corresponding designated pulmonary artery. The performing aspiration of materials may include removing thrombotic materials from the designated pulmonary artery. The performing infusion of fluid may include infusing pharmaceuticals into the designated pulmonary vein, and the performing aspiration of materials may include aspirating the pharmaceuticals and thrombotic materials through the designated pulmonary artery. The performing aspiration of materials may include aspirating blood from the designated pulmonary artery, and the method may further include filtering the blood, and reinfusing the filtered blood into circulations using extracorporeal membrane oxygenation (ECMO) system.

### BRIEF DESCRIPTION OF THE DRAWINGS

The preferred embodiments described herein and illustrated by the drawings hereinafter be to illustrate and not to limit the invention, where like designations denote like elements.

FIG. 1A is a side perspective, cross-sectional view of an embodiment of a transseptal insertion device.

FIG. 1B is a side perspective, cross-sectional view of an embodiment of a transseptal insertion device showing a dilator extending partially through and extending out from device.

FIG. 1C is a side perspective, cross-sectional view of an embodiment of a transseptal insertion device showing a dilator extending partially through the device.

FIG. 2A is a is a perspective view of an embodiment of a transseptal insertion device with hypotube connected to one or more balloons.

FIG. 2B is a is a front view of an embodiment of a transseptal insertion device with hypotube connected to one or more balloons.

FIGS. 2C-2D are side views of embodiments of transseptal insertion device with ultrasound imaging or visualizing capability.

FIG. 3A is a is a perspective view of an embodiment of a transseptal insertion device with multiple balloons and hypotubes connected to the multiple balloons.

FIG. 3B is a is a front view of an embodiment of a transseptal insertion device with multiple balloons and hypotubes connected to the multiple balloons.

FIG. 4 is a perspective, cross-sectional view of an embodiment of a transseptal insertion device with radiofrequency energy capability.

FIG. 5 is a is a perspective view of an embodiment of a transseptal insertion device with a drive assembly coupled to dilator, and knob coupled to the drive assembly.

FIG. 6 is a perspective, cross-sectional view of an embodiment of a transseptal insertion device showing inflated overhanging balloon and dilator positioned within device and subplanar to overhanging balloon.

FIG. 7 is a cross-sectional, end view of an embodiment of a transseptal insertion device and dilator shown prior to puncturing an interatrial cardiac septum with inflated overhanging balloon.

FIG. 8 is a perspective, cross-sectional view of an embodiment of a transseptal insertion device with dilator advanced forward in order to tent an interatrial septum.

FIG. 9 is a perspective, cross-sectional view of an embodiment of a transseptal insertion device with a transseptal wire advanced post-puncture through interatrial septum.

FIGS. 10A-10C are perspective, cross-sectional views of an embodiment of a flexible transseptal insertion device with different angulations.

FIG. 11 is a side view of an embodiment of transseptal insertion device with an overhanging balloon with marking.

FIG. 12 is a side view of an embodiment of transseptal insertion device with an overhanging balloon with a marker band.

FIG. 13 is a cross-sectional side view of an embodiment of a transseptal insertion device that includes a dilator with an electrode tip.

FIG. 14 is a side view of an embodiment of a transseptal insertion device with mechanical deflection capability.

FIG. 15 is side views of embodiments of curved dilators that may be used in embodiments of a transseptal insertion device.

FIG. 16 is a perspective side view of a proximal end of an embodiment of a transseptal insertion device showing a handle and a stabilizer.

FIGS. 17A-17B are side views of an embodiment of a transseptal insertion device with balloons capable of differential inflation.

FIG. 18 is a side view of a malleable or flexible transseptal needle that may be used in embodiments of a flexible transseptal insertion device with multiple angulations.

FIG. 19 shows closed loop catheter system of the disclosed invention isolating desired segment of pulmonary vascular circulation.

FIG. 20 shows a balloon tipped pulmonary venous catheter insertion device in the proximal pulmonary vein via trans septal access.

FIG. 21 shows a close view of the portion Cl of FIG. 20 in which balloon tipped pulmonary venous catheter insertion device cannulates and isolates designated pulmonary vein after balloon at distal tip of the venous catheter insertion device is fully inflated.

FIG. 22 shows a balloon tipped pulmonary artery catheter insertion device cannulating and isolating designated pulmonary artery after distal balloon tip is fully inflated.

FIG. 23 shows a balloon tipped catheter insertion device cannulating the pulmonary artery and isolating designated pulmonary artery after distal balloon is inflated being attached to an aspiration apparatus on its proximal end (outside the body) and aspirating matter through the lumen of the balloon tipped pulmonary artery catheter and a balloon tipped catheter cannulating the pulmonary vein as described in FIG. 20 and FIG. 21 attached to an apparatus that can infuse fluid at a designated pressure and flow rate on its proximal end (outside the body).

FIG. 24A-24B show a side view and a cross-sectional view of the distal tip portion of the balloon tipped catheter insertion device of the disclosed invention.

FIG. 25 shows a flowchart diagram for a method of using the closed loop catheter system that includes a first balloon tipped catheter insertion device and a second balloon tipped catheter insertion device for pulmonary embolism.

### DETAILED DESCRIPTION

In this section, some embodiments of the invention will be described more fully with reference to the accompanying drawings, in which preferred embodiments of the invention are shown. This invention, however, may be embodied in many different forms and should not be construed as limited to the embodiments set forth herein. Rather, these embodiments are provided so that this disclosure will be thorough and complete, and will convey the scope of the invention to those skilled in the art. Like numbers refer to like elements throughout, and prime notation is used to indicate similar elements in alternative embodiments. Parts that are the same or similar in the drawings have the same numbers and descriptions are usually not repeated.

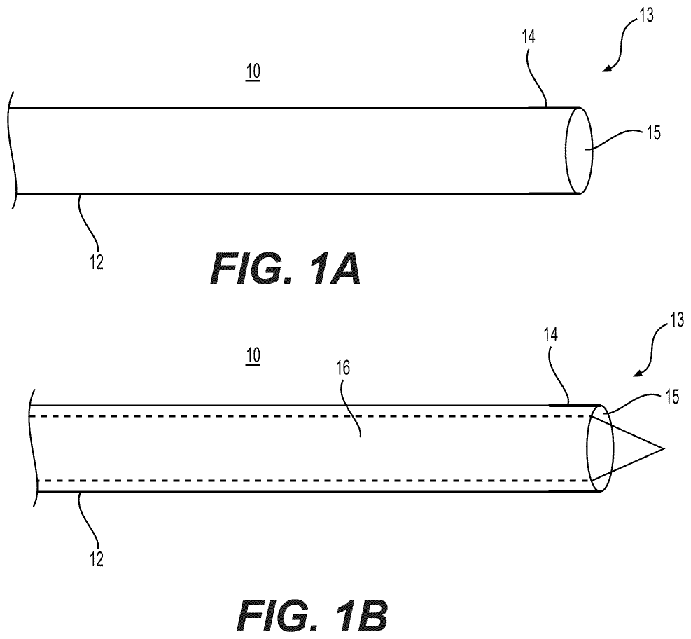

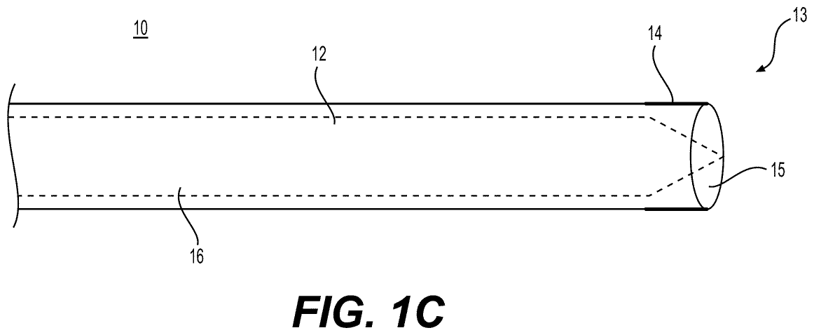

With reference to FIGS. 1A-1C, shown is an embodiment of balloon tipped catheter insertion device 10. Shown is the distal end of balloon tipped catheter insertion device 10, i.e., the end of transseptal insertion device 10 with opening through which dilator, catheter, and needle may extend, e.g., to puncture interatrial cardiac septum. As shown in FIG. 1A, catheter insertion device 10 includes outer sheath or balloon shaft 12 and one or more balloons 14 located at distal tip 13 of catheter insertion device 10. Sheath 12 may contain and define a center lumen 15. Sheath 12 may be fabricated from various materials, including, e.g., polymers, including thermoplastics elastomers (TPEs) such as PEBA (e.g., Pebax®), nylons, thermoplastic polyurethanes (TPUs) such as Pellathane®, similar materials and combinations thereof. Sheath 12 may be referred to as catheter shaft and used in cardiac catheterizations. After puncture, sheath 12 may be inserted through septum into left atrium. Alternatively, sheath 12 may contain a separate catheter that is inserted through septum post puncture. Catheter insertion device 10 also includes dilator 16, positioned in center lumen 15, as shown in FIG. 1B. The one or more balloons 14 are preferably sealed, air-tight and water-tight, on both its ends to sheath 12.

With continuing reference to FIG. 1A, in view shown, overhanging one or more balloons 14 are uninflated. Although cross-section of balloons 14 shown on top and bottom of distal tip 13, balloons 14 preferably extend around circumference of distal tip or end 13 of catheter insertion device 10. Overhanging one or more balloons 14 are of form such that balloons 14 overhang or extend from distal tip 13 of sheath 12 when inflated.

In FIG. 1B, dilator 16 is shown positioned within and partially extending out of sheath 12, past distal tip 13 of device 10. Overhanging one or more balloons 14 are uninflated and dilator 16 extends past balloons 14. It is noted that the relative sizes of sheath 12 and dilator 16 shown are for illustrative purposes as the diameter of dilator 16 may be relatively larger or smaller than shown in relation to the diameter of sheath 12, although dilator 16 necessarily has a smaller diameter than sheath 12. Although dilator 16 is shown to have a pointed end, dilator 16 may have a rounded or relatively flat end. Embodiments, as described herein, are designed and intended to puncture septum without use of a needle or other sharp instrument.

With reference now to FIG. 1C, dilator 16 is shown positioned within lumen 15 of sheath 12. Tip of dilator 16 is positioned within distal tip 13 of catheter insertion device 10 sub-planar to end of catheter insertion device 10. The position of dilator 16 may be in immediately prior to inflation of one or more balloons 14. It is noted that the relative sizes of catheter/sheath 12 and dilator 16 shown are for illustrative purposes as the diameter of dilator 16 may be relatively larger or smaller than shown in relation to the diameter of sheath 12. Ordinarily, dilator 16 has smaller diameter or gauge then catheter/sheath 12, although fit of dilator 16 in catheter/sheath 12 is preferably snug enough so that dilator 16 does not move (laterally or axially) relative to position or “wobble” within transseptal insertion device 10. Dilator 16 necessarily has a smaller diameter than sheath 12. In embodiments, sheath 12 material may be sufficiently malleable to enable larger diameter dilators 16, and other larger diameter devices, to be passed through sheath 12. In such embodiments, sheath 12 will stretch to accommodate the larger diameter dilator 16 or other device.

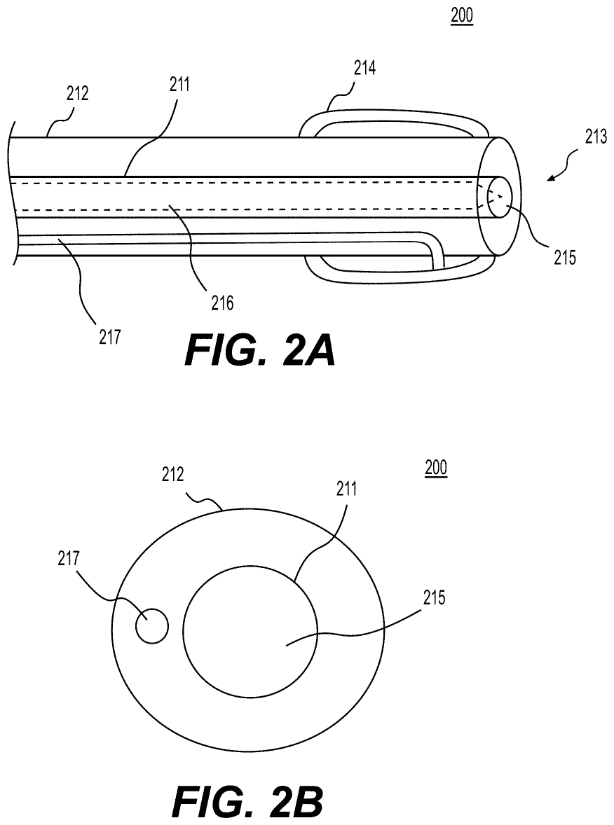

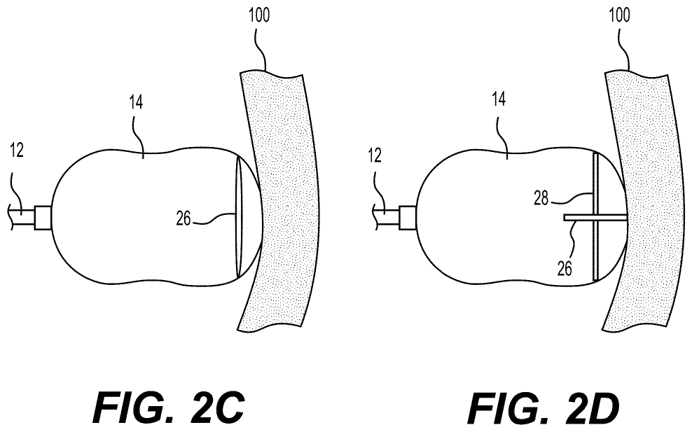

With reference to FIG. 2A, shown is a side perspective view of an embodiment of catheter insertion device 200. Shown is the distal end of catheter insertion device 200, i.e., the end of catheter insertion device 200 with opening through which dilator, catheter, and needle may extend, e.g., to puncture interatrial cardiac septum. As shown in FIG. 2A, catheter insertion device 200 includes outer sheath or catheter shaft 212 and one or more balloons 214 located at distal tip 213 of catheter insertion device 200. Sheath 212 may contain lumen shaft 211 that defines center lumen 215. Sheath 212 may be fabricated from various materials, including, e.g., polymers, including thermoplastics elastomers (TPEs) such as PEBA (e.g., Pebax®), nylons, thermoplastic polyurethanes (TPUs) such as Pellathane®, similar materials and combinations thereof. Sheath 212 may be referred to as catheter shaft and used in cardiac catheterizations. After puncture, sheath 212 may be inserted through septum into left atrium. Alternatively, sheath 212 may contain multiple lumen shafts that define multiple lumens separately. Transseptal insertion device 200 also includes dilator 216, positioned in center lumen 215. The one or more balloons 214 are preferably sealed, air-tight and water-tight, on both their ends to sheath 212. Catheter insertion device 200 includes hypotube 217 for inflation or deflation of one or more balloons 214. Hypotube 217 may be contained in sheath or catheter shaft 212. Catheter insertion device 200 may further include a port (not shown) connected to hypotube 217 to supply gas or fluid to inflate one or more balloons 214, or to remove gas or fluid from one or more balloons 214 to deflate balloons 214. Balloons 214 may be fully inflated or deflated, or may be inflated or deflated as much as desired. With reference to FIG. 2B, shown is a front, cross-sectional view of distal end 213 of the embodiment of transseptal insertion device 200 that shows cross-sectional views of sheath 212, center lumen 215, and hypotube 217.

In the embodiment shown in FIGS. 2A-2B, catheter insertion device 200 may include ultrasound chips or transducers 26 for ultrasound imaging or visualizing (see FIGS. 2C-2D). The transseptal sheath 212 or balloon 214 may house (inside or on) an ultrasound chip or transducer which may be used to guide the insertion procedure. Ultrasound chip or transducer emits and receives ultrasound energy, that may be detected by known ultrasound visualization devices, to create an image of the cardiac chambers (e.g., the right atrium, fossa, interatrial septum, left atrium, atrial appendage, mitral valve, ventricle, etc.). Ultrasound chips and transducers are transducers that convert ultrasound waves to electrical signals and/or vice versa. Those that both transmit and receive may also be called ultrasound transceivers; many ultrasound sensors besides being sensors are indeed transceivers because they can both sense and transmit. Such imaging will allow the operator(s) of catheter insertion device 200 to visualize the cardiac chambers and the determine the location of the distal end or tip 213 of catheter insertion device 200, enabling more precise operation of catheter insertion device 200. Such a ultrasound chips or transducers used may be similar to ultrasound chip or transducer described in US Pat. App. Pub. 2003/019546, which is herein incorporated by reference, or any other ultrasound transducer known to those of ordinary skill in the art that may be fabricated on scale small enough to be deployed on or in sheath 212 or balloon 214.

With reference to FIGS. 2C-2D, shown are embodiments of catheter insertion device 200 with ultrasound imaging or visualizing capability. Balloon 14 shown includes one or more ultrasound chips or transducers 26 deployed in or on balloon 14. Ultrasounds chips or transducers 26 may be ultrasound transceivers that both emit and receive waves, convert the ultrasound waves to electrical signals, transmit the electrical signals, e.g., through a wire that runs via sheath 12. Ultrasounds chips or transducers 26 may be connected via WiFi or other wireless connection, to an external imaging device that produces images from the received signals (both still and video images).

Ultrasound chips or transducers 26 may be affixed to interior or exterior surface of balloon 14. Ultrasound chips or transducers 26 may be arranged in a line, disc, or cross-shape. Ultrasound chips or transducers 26 may be arranged to be forward facing (e.g., on distal end of balloon facing towards interatrial septum), as shown in FIG. 2C, or in a different direction/orientation, such as sideways and forward facing (e.g., facing towards interatrial septum and facing perpendicular to the distal or front end), as shown in FIG. 2D. Indeed, orientation of ultrasound chips or transducers 26 may depend on whether balloon 14 is inflated or not. When balloon 14 is fully inflated, as shown in FIG. 2C, ultrasound transducer 26 may be forward facing (or forward and perpendicularly facing as shown in FIG. 2D). However, when balloon 14 is deflated, ultrasound transducer 26 may be folded flat and positioned on side of distal tip 13 of sheath 12. Hence, when balloon 14 is deflated, ultrasound chip or transducer 26 may be side-facing. During inflation ultrasound transducer 26 orientation will change as balloon 14 inflates (moving from side-facing orientation to forward facing orientation with the ultrasound transducer 26 shown in FIG. 2C). Accordingly, operator(s) of transseptal insertion device 200 may vary the inflation of balloon 14 to achieve different orientations of ultrasound transducer 26 for different imaging views.

Ultrasound chip or transducers 26 may emit and/or receive/detect ultrasound waves that may be reflect off of surfaces and structures, e.g., within atrium, and then read by imaging system (not shown), e.g., connected to ultrasound chips or transducers 26 via wire or cable extending through, e.g., lumen 15 in sheath 12. In this manner, ultrasound chips or transducers 26 may enable visualization of the interatrial septum and the left atrial structures.

It is also noted that ultrasound chips or transducers 26 may be deployed on distal tip 13 of sheath 12 (or elsewhere on or in sheath 12). Ultrasound chips or transducers 26 may be installed or configured to be forward facing (facing towards distal end of sheath 12). Alternatively, ultrasound chips or transducers 26 may be flipped to be rear facing (facing towards proximal end of sheath 12). Varying orientations of ultrasound chips or transducers 26 may be implemented.

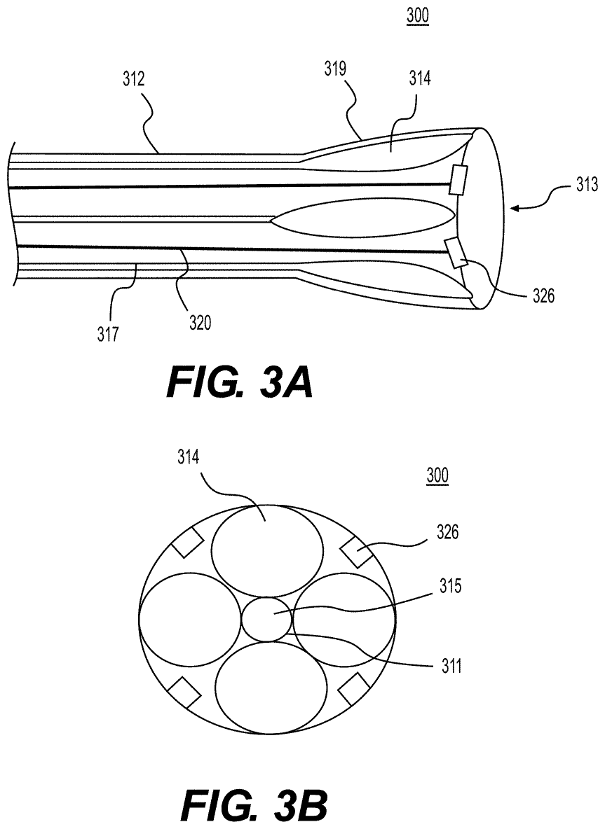

With reference to FIGS. 3A-3B, shown is catheter insertion device 300 including multiple balloons 314, which surround center lumen shaft 311 that defines center lumen 315, and sheath or catheter shaft 312 that includes center lumen shaft 311 and hypotubes 317 connected to multiple balloons 314. FIG. 3A is a side view of sheath or catheter shaft 312, and FIG. 3B is a front cross-sectional view of sheath or catheter shaft 312. Balloons 314 are in various shapes such as round, cylindrical, spherical, tear drop shaped or pear shaped, and are in various lengths. Balloons 314 may be with or without overhang over shaft. Balloons 314 are positioned around distal tip or end 313, and may extend around circumference of distal tip or end 313. Multiple balloons 314 are connected to one or more hypotubes 317, and inflated or deflated via hypotubes 317 that are contained in sheath or catheter shaft 312. Each of balloons 314 may be connected to corresponding hypotube 317 to independently control the inflation and deflation of balloons 314. Alternatively, balloons 314 may share one or more hypotubes 317. Inflation fluid or gas may flow through hypotubes 314 to inflate or deflate balloons 314. Outer covering 319 may cover the multiple balloons 314.

In between balloons 314, there are one or more ultrasound chips or transducers 326 that provide ultrasound imaging or visualizing capability. For illustrative purposes, FIG. 3B shows ultrasound chips or transducers 326 disposed between balloons 314, but ultrasound chips or transducers 326 may be deployed in or on balloons 314. Ultrasound chips or transducers 326 may be affixed to interior or exterior surface of balloon 314. Ultrasounds chips or transducers 326 may be ultrasound transceivers that both emit and receive waves, convert the ultrasound waves to electrical signals, transmit the electrical signals, e.g., through wire 320 that runs inside sheath or catheter shaft 312. However, ultrasound chips or transducers 326 may be connected wirelessly via WiFi or other wireless connection, to an external imaging device that produces images from the received signals (both still and video images).

Ultrasound chips or transducers 326 may be designed in the shape of the balloons 314. The balloons 314 may be round, cylindrical, spherical, tear drop shaped or pear shaped with overhang or without overhang. Ultrasound chips or transducers 326 may have shapes corresponding to the shapes of balloons 314. Alternatively, one or more ultrasound chips or transducers 326 may be deployed in a shape corresponding to the shapes of balloons 314. Depending on the shapes of balloons 314, ultrasound chips or transducers 326 may be side facing, front facing or back facing. Ultrasound chips or transducers 326 may be arranged in a line, disc, or cross-shape. Ultrasound chips or transducers 326 may be arranged to be forward facing (e.g., on distal end of balloon facing towards interatrial septum), or in a different direction/orientation, such as sideways and forward facing (e.g., facing towards interatrial septum and facing perpendicular to the distal or front end).

Orientations of ultrasound chips or transducers 326 may depend on whether balloons 314 are inflated or not. When balloons 314 are fully inflated, ultrasound chips or transducers 326 may be forward facing. However, when balloons 314 are deflated, ultrasound chips or transducer 326 may be folded flat and positioned on side of distal tip 313 of center lumen 315. Hence, when balloons 314 are deflated, ultrasound chips or transducer 326 may be side-facing. During inflation, orientation of ultrasound chips or transducers 326 may change as balloons 314 inflate (moving from side-facing orientation to forward facing orientation). Accordingly, operator(s) of catheter insertion device 300 may vary the inflation of balloons 314 to achieve different orientations of ultrasound chips or transducers 326 for different imaging views.

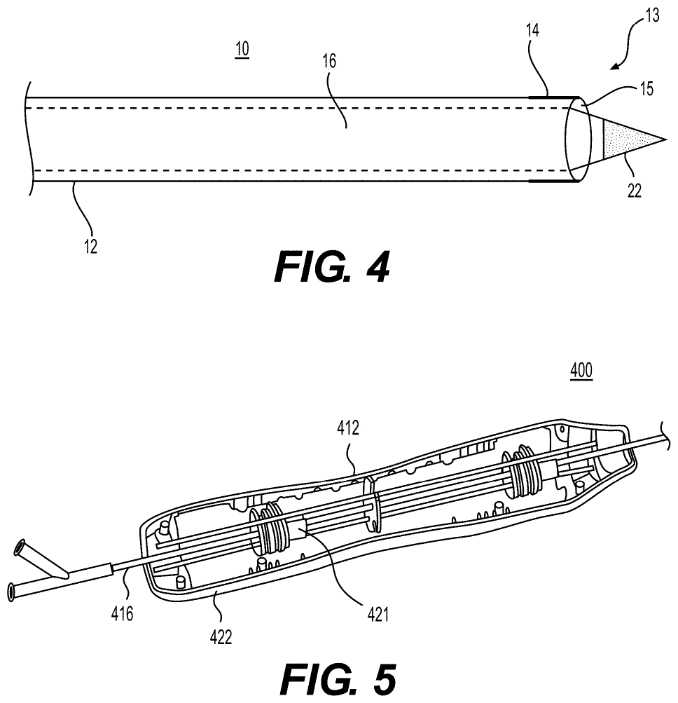

With reference now to FIG. 4, shown is an embodiment of catheter insertion device 10 with radiofrequency (RF) energy capability. Catheter insertion device 10 shown includes sheath 12, overhanging one or more balloons 14, and dilator 16. Dilator 16 may include cap or crown 22, on distal end as shown, with RF energy capability or capable of delivering RF energy. Alternatively, cap or crown may include or be an RF electrode. Dilator 16 may be connected, e.g., on proximate end (not shown) to a radiofrequency energy source (not shown) at, e.g., external hub, that provides RF energy to cap or crown 22. The RF energy may be delivered through dilator 16. So equipped with cap or crown 22, dilator 16 may tent interaxial septum and create puncture of interaxial septum through delivery of RF energy. In this embodiment, the use of a sharp needle may be avoided. The dilator with cap or crown on distal end with RF energy capability or capable of delivering RF energy may be used for transseptal insertion devices 200 and 300 shown in FIGS. 2A-2B and 3A-3B.

With reference to FIG. 5, shown is catheter insertion device 400 including drive assembly 421, which is coupled to dilator 416, and knob 422 coupled to drive assembly 421 to cause dilator 416 to traverse along an axial direction of sheath or catheter shaft 412. Dilator 416 may move backwards or forwards along the axial direction of sheath 412 while knob 422 is rotated. The drive assembly 421 may include nut assembly to drive the dilator 416. Dilator 416 may be with or without RF energy capability.

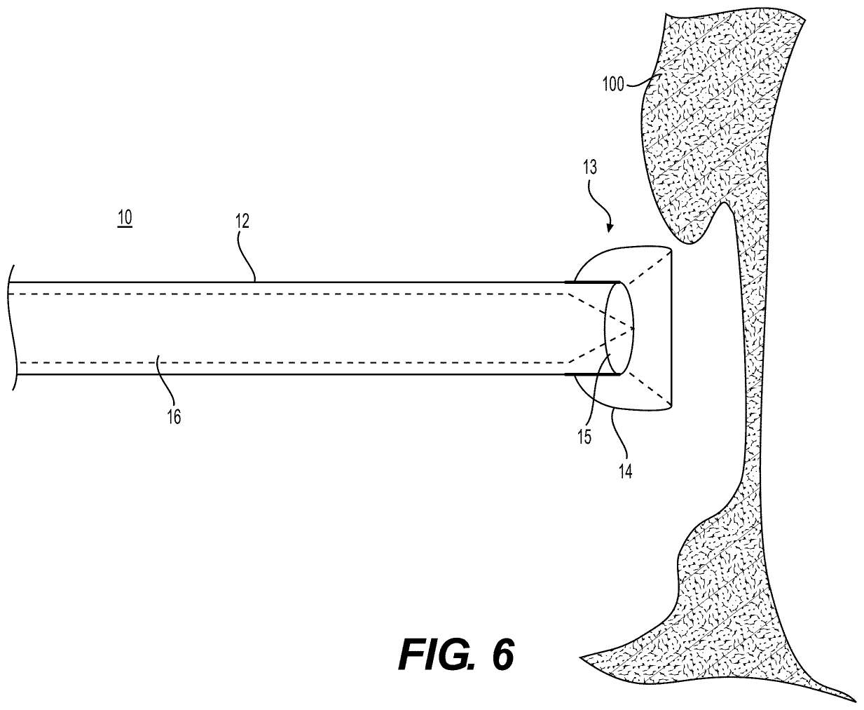

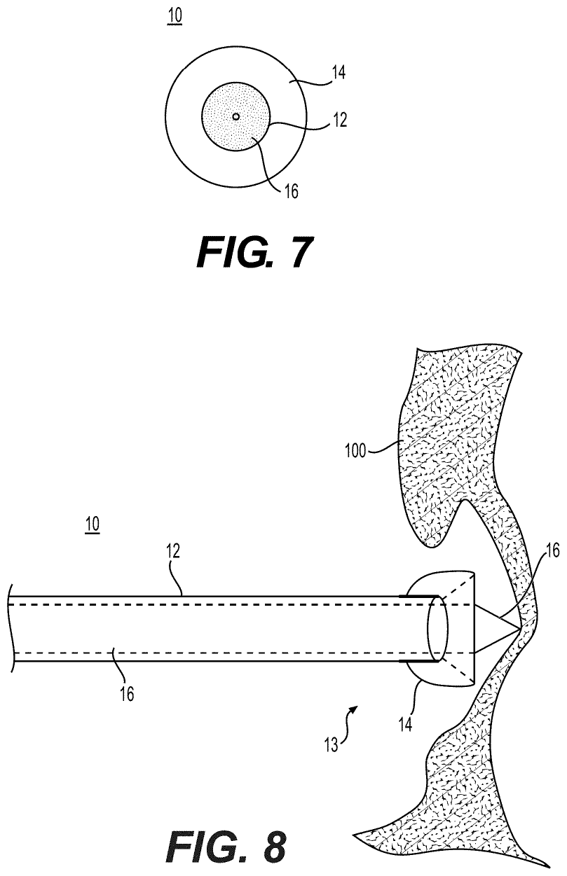

With reference now to FIG. 6, shown is distal end of an embodiment of catheter insertion device 10 in which overhanging balloons 14 is inflated by supplying gas or fluid into balloon 14 through hypotube (not shown). Dilator 16 is shown positioned within center lumen 15 of sheath 12 with tip of dilator 16 positioned at distal tip 13 of catheter insertion device 10 and sub-planar to overhanging balloon 14. The plane that is referred to here is the plane perpendicular to the axis of catheter insertion device 10 and dilator 16, formed by the end of overhanging balloon 14. Hence, dilator 16 remains sub-planar to overhanging balloon 14 until operator intends balloon 14 to be deflated and dilator 16 to tent and puncture interatrial septum 100. As noted above, balloon 14 preferably extends completely around circumference of tip 13 of catheter insertion device 10. Accordingly, FIG. 7 only illustrates cross-section of inflated balloon 14.

With reference now to FIG. 7, shown is a front, cross-sectional view of distal end an embodiment of catheter insertion device 10 in which overhanging balloon 14 is inflated. As shown, inflated overhanging balloon 14 preferably extends around entire circumference of sheath 12 (and, therefore, device 10). Shown situated within lumen 15 of sheath 12 is tip of dilator 16. Tip of dilator 16 is positioned within tip 13 of catheter insertion device 10, as it would be prior to being extended past tip 13 and puncturing an interatrial cardiac septum.

With reference now to FIG. 8, shown is distal end of an embodiment of transseptal insertion device 10 with dilator 16 advanced forward in order to tent the interatrial septum 100. Dilator 16 is shown extending through center lumen 15 of sheath 12 and past overhanging balloon 14. At this stage, balloon 14 may be deflated by removing gas or fluid in balloon 14 through hypotube. Extended as such, and pressed against interatrial septum 100, dilator 16 tents the interatrial septum 100 away from catheter insertion device 10.

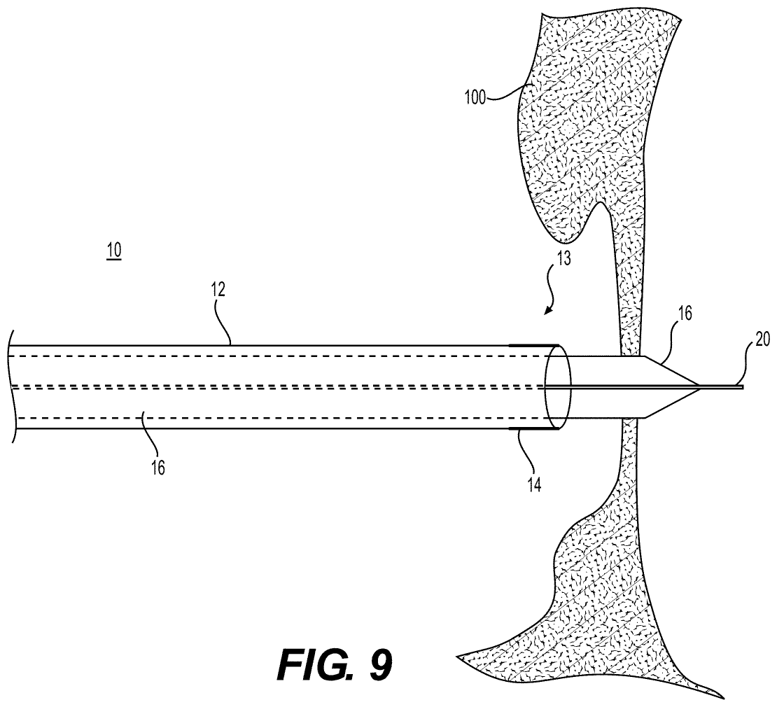

With reference now to FIG. 9, shown is shown is distal end of an embodiment of catheter insertion device 10 with dilator 16 advanced forward through interatrial septum 100, after puncturing septal wall (e.g., through application of energy through dilator 16 as described herein) and transseptal wire or wire rail 20 extending through dilator 16 and into left atrium chamber 110. Wire rail 20 may sit in a lumen 19 of dilator 16. Dilator 16 may be used as a conduit to advance the wire rail 20 into the left atrium.

Wire rail 20 may act as a guide for devices to enter the left atrium through the puncture in the septal wall made by transseptal insertion device 10. For example, wire rail 20 may guide catheter insertion device 10 or other catheters in the left atrium. In this manner, catheters may be advanced safely into the left atrium over or guided by wire rail 20. In an embodiment, wire rail 20 may be energized (e.g., to ablate or puncture the septum with energy delivered from source at proximal end of catheter insertion device 10).

With continued reference to FIG. 9, dilator 16 preferably defines and includes an opening or lumen 19 extending through its tip and through which transseptal wire 20 extends. With dilator 16 extended as shown and tenting interatrial septum, septum may be punctured by energy delivered through cap or electrode at tip of dilator 16 and transseptal wire rail 20 extended through opening in tip of dilator 16 and through puncture made in interatrial septum by dilator 16 cap.

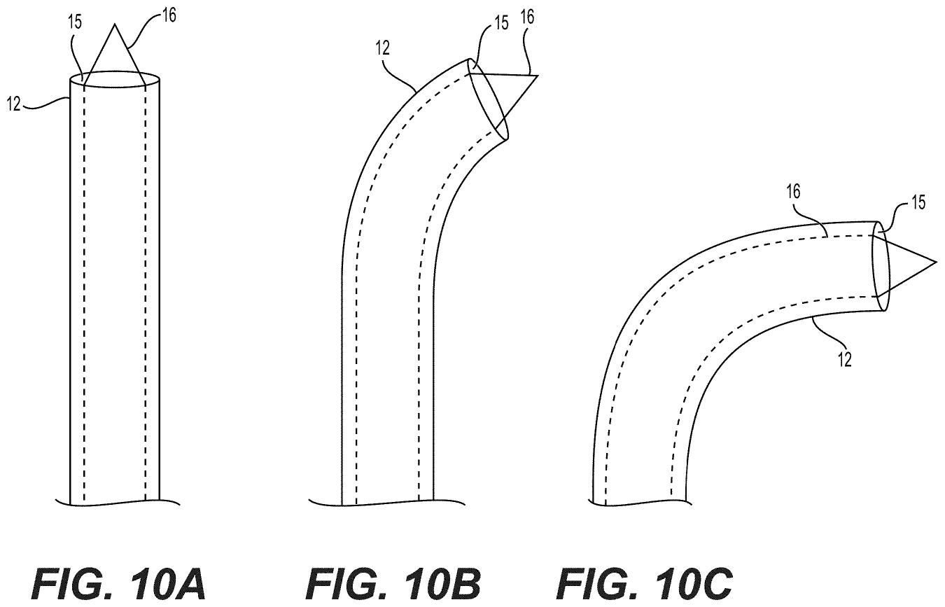

With reference to FIGS. 10A-10C, shown are different views of an embodiment of catheter insertion device 10 with a flexible sheath 12 flexed or angulated at different angles. Catheter insertion device 10 may be flexed or angulated depending on the anatomy of the atria using fixed angled dilators 16 that are inserted into lumen shaft of sheath 12, causing sheath 12 to flex. Such fixed angled dilators 16 may be, e.g., any angle from 0-270°. Alternatively, sheath 12, lumen shaft and dilator 16 may be all flexible (preferably, hypotubes, needle and catheter inserted through such flexible sheath 12 are flexible or malleable, at least in part) and catheter insertion device 10 may be flexed or angulated, thereby flexing or angulating sheath 12 and dilator 16, using, e.g., a handle or wire (not shown) connected to tip 13 of device 10. Handle and/or wire may also be used to turn or flex or move tip 13 of catheter insertion device 10, e.g., moving tip 13 of sheath “up” or “down” or “left” or “right” or angulating tip 13 relative to axis of sheath 12 as shown.

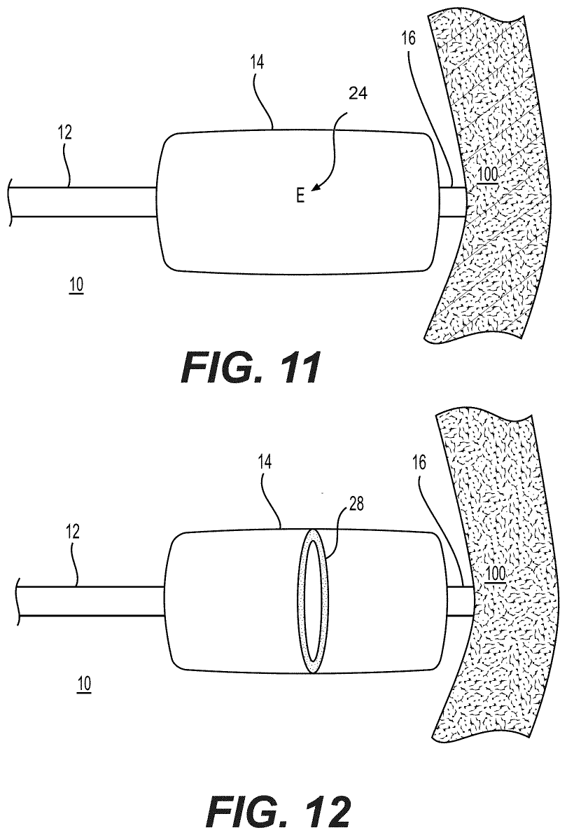

With reference now to FIG. 11, shown is distal end of an embodiment of catheter insertion device 10 with inflated overhanging balloon 14. Balloon 14 shown is an embodiment with one or more markers 24. Marker 24 may be, e.g., a radiopaque and/or echogenic marker 24. As a radiopaque or echogenic marker, marker 24 will be visible on scanners used by those performing cardiac catheterizations. The markers 24 may be in the form of letters, such as an E or a C. Marker 24 enables the appropriate positioning of balloon 14 and sheath 12 in the 3-dimensional space (e.g., of the atrium) using imaging to view the marker 24 and, therefore, the position of balloon 14.

Specifically, in operation, the less posterior distal tip 13 is positioned, the more of the E (or C) will be shown. As operator of transseptal insertion device 10 turns or rotates distal tip 13 toward posterior of patient, less of the arms of the E will be seen. In a preferred embodiment, when only the vertical portion of the E is visible (i.e., appearing as an I) distal tip 13 will be rotated to its maximum posterior position.

With continuing reference to FIG. 11, balloon 14 is shown as inflated. However, distal end of dilator 16 is shown extruding or extending distally from balloon 14, past plane formed by distal end of inflated balloon 14. According, dilator 16 has been moved into the tenting and puncturing position, adjacent to interaxial septum. At this stage, balloon 14 may be deflated or will soon be deflated, and puncture of the interaxial septum is imminent.

With reference now to FIG. 12, shown is another embodiment of overhanging balloon 14 which may be deployed in embodiments of catheter insertion device 10. Overhanging balloon 14 may include ring or band 28 around a portion of balloon 14. Ring or band 28 may serve as a marker, similar to markers 24 shown in FIG. 11. Hence, ring 28 may be radiopaque or echogenic and may be view by scanning devices used for visualization in cardiac catheterizations (e.g., fluoroscopic imaging devices). Similar to the letter E or C, the view of the ring 28 changes as the distal tip 13 of transseptal insertion device 10 moves more posterior. When in a least posterior position, ring 28 may appear as just a line or band positioned across axis of catheter insertion device 10. When device 10 is rotated so that distal tip 13 is significantly closer to the posterior, ring 28 may appear as a full “flat” circle or ring. In FIG. 12, distal tip 13 is partially rotated so that ring 28 is partially visible.

With reference to both FIGS. 11 and 12, the marker 24 and ring 28 are described and shown as located on balloon 14. In embodiments, marker 24 and/or ring 28 may also be located on sheath 12 and/or dilator 16. So located, marker 24 and/or ring 28 would operate in effectively the same manner as described above (i.e., the arms of the E would disappear as the distal end was moved more to the posterior and the ring would become more visible). Markers 24 and/or rings 28 may be placed on all of balloon 14, sheath 12, and dilator 16, or a combination thereof.

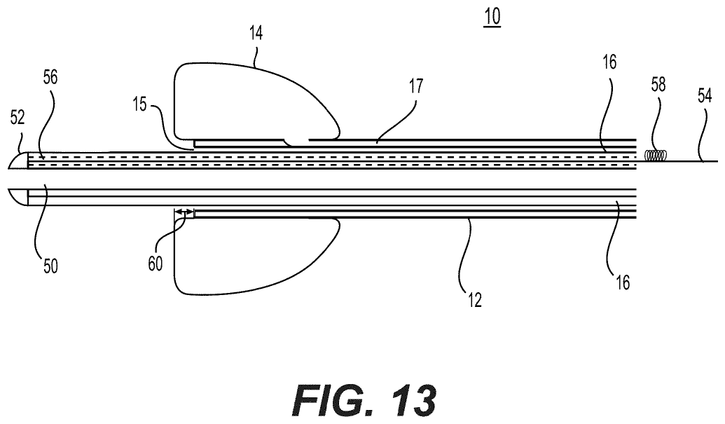

With reference now to FIG. 13, shown is distal end of an embodiment of transseptal insertion device 10 that includes dilator 16 with electrode tip. Shaft of dilator 16 defines and contains a center lumen 50. Lumen 50 may be defined in the range of, but not limited to, 0.020 to 0.040 inches. Dilator 16 may be made from a polymer material (e.g., HDPE, LDPE, PTFE, or combination thereof). Dilator shaft 16 shown includes a distal electrode tip 52. Electrode tip 52 may be comprise a metallic alloy (e.g., PtIr, Au, or combination thereof). In preferred embodiments, the size and shape of electrode tip 52 is selected to be sufficient to generate a plasma for in vivo ablation of tissue in an applied power range of, but not limited to, 20-30W. Electrical conductor 54 extends from electrode tip 52 to the proximal end (not shown) of the dilator 16. Electrical conductor 54 may run axially through an additional lumen 56 defined by and contained in dilator shaft 16. Electrical conductor 54 may contain a coil feature 58 to accommodate lengthening during bending or flexing of dilator 16.

Attached to distal end of sheath 12 is contains overhanging balloon 14 that is connected to hypotube 17. Overhanging balloon 14 may be made from a polymer material (e.g., PET, Nylon, Polyurethane, Polyamide, or combination thereof). Overhanging balloon 14 may be in the range of, but not limited to, 5-20 mm in diameter and 20-30 mm in length. Overhanging balloon 14 may be inflated via injection of gas or fluid through hypotube 17 connected to balloon 14. Overhanging balloon 14 may be deflated by removing gas or fluid in balloon 14 through hypotube 17 connected to balloon 14. During the proper functioning or operation of catheter insertion device 10 for puncturing the interatrial septum, balloon 14 may be deflated when dilator 16 moves out of lumen 15 by removing gas or fluid from balloon 14. Overhanging balloon 14 is of form such balloon 14 overhangs or extends from distal end 13 of sheath 12. Overhang or extension 60 may be in the range of, but not limited to, 0.0 mm-5.0 mm. The end of the overhang or extension 60 is the plane to which dilator 16 remains sub-planar until moving to tent and puncture the interatrial septum.

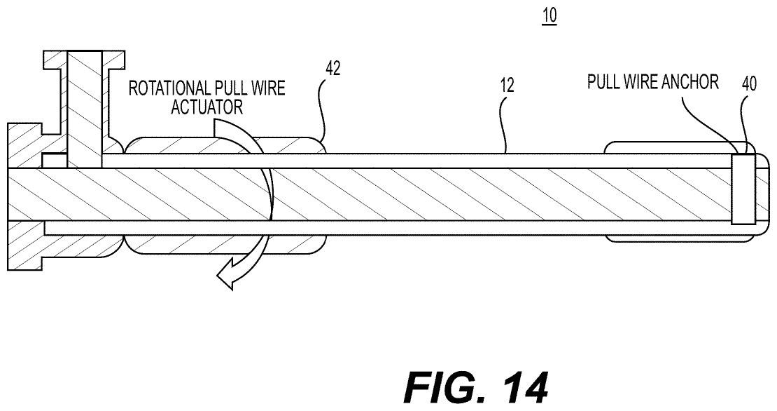

With reference now to FIG. 14, shown is an embodiment of catheter insertion device 10 that includes a mechanical deflection mechanism. Mechanical deflection mechanism may enable distal end of sheath 12 to be deflected or angulated to various angles with respect to axis of catheter insertion device 10. Mechanical deflection mechanism may include a pull wire anchor 40 affixed to distal end of sheath 12 and pull wire actuator 42 connected to pull wire anchor 40 with pull wire (not shown). Rotation of pull wire actuator 42, as shown, may exert force on pull wire anchor 40 that deflects or angulates distal end of sheath 12. Pull wire actuator 42 may be rotated by handle connected thereto (not shown). Deflection or angulation of distal end of sheath 12 may enable better intersection (e.g., more perpendicular, flush) with interaxial septum and, therefore, better puncture and insertion by catheter insertion device 10.

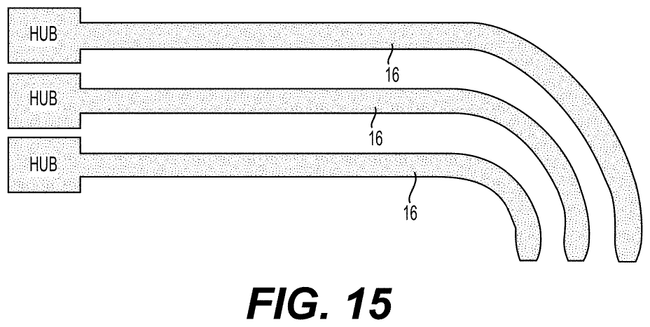

With reference now to FIG. 15, shown are three (3) embodiments of curved dilators 16, each with a different curve profile (i.e., different angle of deflection or curve). Curved dilators 16 may be used in embodiments of catheter insertion device 10 with flexible or malleable sheath 12. Such a flexible or malleable sheath 12 may be referred to as a steerable sheath 12 as it is “steered” by curved dilator 16 inserted in sheath 12.

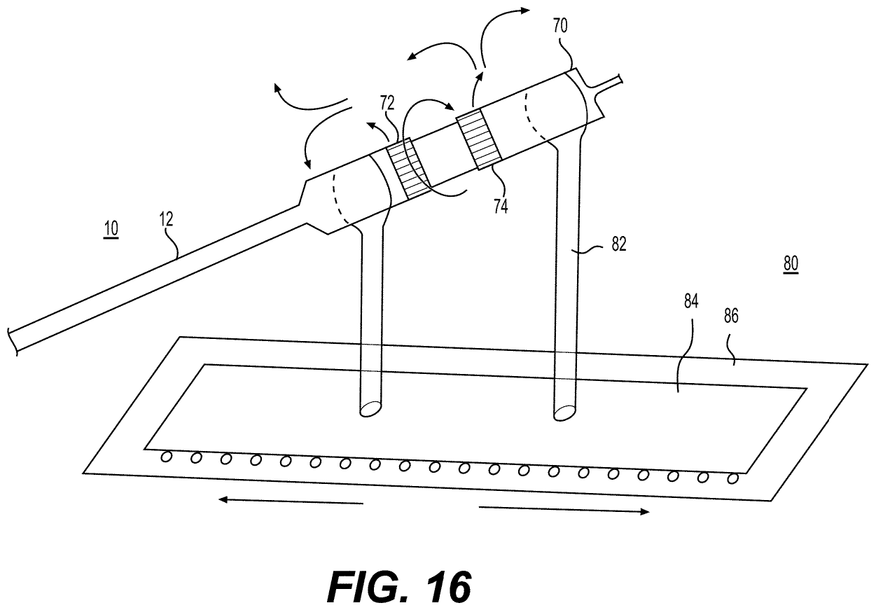

With reference now to FIG. 16, shown is an embodiment of catheter insertion device 10 with an external stabilizer 80. Stabilizer 80 keeps proximal end of catheter insertion device 10 stable while allowing movement of catheter insertion device 10 towards the distal and proximal ends of device 10, rotational/torqueing movement of proximal end of device 10, and manipulation of dials or other controls of device 10. In effect, stabilizer 80 substantially prevents unwanted movement of the catheter insertion device 10 and, importantly, distal end of sheath 12, balloon 14, and dilator 16.

Stabilizer 80 includes connecting rods or arms 82 that connect stabilizer 80 to handle 70 at proximal end of transseptal insertion device 10. Connecting arms 82 are attached to stabilizer platform 84. Connecting arms 82 preferably hold the handle 70 securely and tightly, while permitting desired rotational movements and control manipulation. Stabilizer platform 84 is moveably attached to stabilizer base 86 so that stabilizer platform 84, and hence handle 70 and catheter insertion device 10, may be slid forwards and backwards along axis of catheter insertion device 10 towards and away from insertion point in patient (typically femoral vein at the groin of patient). Stabilizer base 86 is typically secured to a flat, stable surface, such as a table, or the leg of the patient. Configured as such, stabilizer 80 prevents unwanted vertical, rotational, or other movement of catheter insertion device 10 and its handle 70, keeping transseptal insertion device 10 and its handle 70 stable while permitting precise manipulation of handle 70 and its controls.

With continuing reference to FIG. 16, as shown, proximal end of catheter insertion device 10 may include a handle 70 for control and manipulation of catheter insertion device 10 and, particularly, dilator 16 and distal end of dilator 16. Handle 70 may include a dial 72 that may be used to turn or deflect distal end of dilator 16, effectively moving the distal end of dilator 16 up or down in relation to axis of transseptal insertion device 10 (as indicated by arrows in FIG. 16). Handle 70 may also include dial 74 for extruding/extending distal end of dilator 16 out of sheath 12 and retracting dilator 16 back into sheath 12, effectively moving dilator 16 along axis of transseptal insertion device 10 (as indicated by arrows in FIG. 16). Handle 70 may also be rotated, as indicated by rotational arrow in FIG. 16, in order to deflect or turn distal end of transseptal insertion device to left or right in relation to axis of catheter insertion device 10, increasing or decreasing dilator 16 angle of deflection in that direction. If dial 72 moves distal end of dilator 16 along Y axis, and catheter insertion device 10 axis is considered the Z axis, so that dial 74 moves dilator 16 along Z axis rotating handle 70 moves distal end of catheter insertion device 10 (and hence distal end of dilator 16) along X axis. Handle 70 includes a port through which dilator 16 and other devices inserted into catheter insertion device 10 may be inserted. Handle 70 may also include one or more tubes or other ports permitting connection to external hubs and external energy sources, inflation liquids or gas.

In embodiments shown herein, balloon 14 and dilator 16 may be used as energy sources in the left atrium and may be used to deliver energy to the pulmonary veins, left atrial appendage, mitral valve and the left ventricle present in the left atrium. Such embodiments may include external energy sources connected to balloon 14 and/or dilator 16 through wires or other conductors extending lumen in sheath 12. Delivery of energy via balloon 14 or dilator 16 may be thermal/Cryo or radiofrequency, laser or electrical. The delivery of such energy could be through a metallic platform such as a Nitinol cage inside or outside balloon 14. Transseptal insertion device 10 may also include an energy source external to the proximal end of the sheath and operatively connected to balloon 14 to deliver energy to balloon 14.

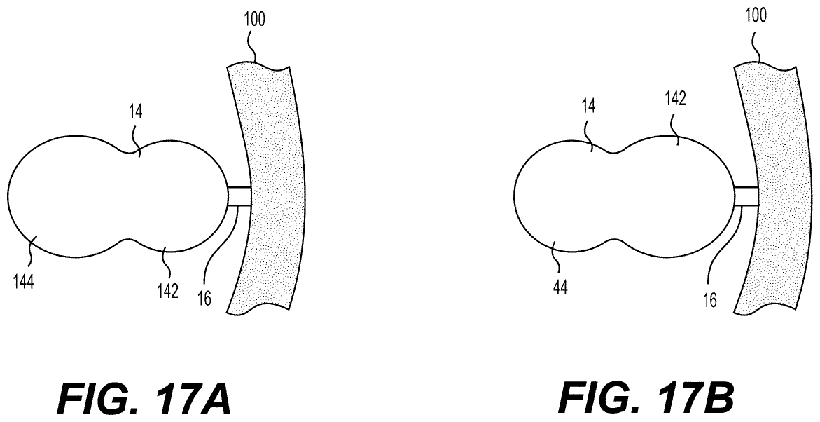

With reference now to FIGS. 17A-17B shown is an embodiment of catheter insertion device 10 enabling differential expansion of balloon 14. Differential expansion of balloon 14 enables balloon 14 inflation to be adjusted based on the needs of the device operator and the conditions present in the patient's heart. For example, the size of the fossa ovalis portion of the interatrial septum may dictate the desired size of the inflated balloon 14 needed at the puncture site (interatrial septum if often punctured through the fossa ovalis). Fossae can vary greatly in size. The larger the fossa, the harder it will be to tent the interatrial septum with balloon 14. Large fossa tend to be saggy and more difficult to manipulate. Hence, with a large fossa, a larger distal end of balloon 14 will make proper tenting of the interatrial septum easier. Indeed, it may be ideal to have balloon 14 inflated uniformly until intersecting or passing through fossa and then differentially expanding distal end 142 of balloon 14 to move fossa out of the way. In FIG. 17A, distal end or portion 142 of balloon 14 is smaller (less expanded) than proximal end 144 of balloon 14.

Oppositely, the smaller the fossa, the easier it will be to tent the interatrial septum but, there will be less room to maneuver balloon 14 near interatrial septum. Consequently, a smaller distal end of balloon 14 is desired. It also may be beneficial to expand the proximal portion 144 more in order to help fix or secure balloon 14 in place. In FIG. 17B, distal end or portion of balloon 14 is larger (more expanded) than proximal end or portion of balloon 14. In both FIGS. 17A and 17B, dilator 16 has extruded from sheath 12 and past distal end of balloon 14, tenting interatrial septum 100, and puncture is imminent.

This differential expansion of balloon 14 may be achieved, e.g., by using different materials for different portions of balloon 14 (e.g., a more expandable material for distal end 142 than proximal end or portion 144, or vice versa). In general, balloon 14 may be made of either compliant or non-compliant material, or a combination thereof. Compliant material will continue expanding as more inflating liquid or gas is added to balloon 14 (at least until failure). Non-compliant material will only inflate up to a set expansion or designated inflation level. Combinations of compliant and non-compliant material may be used to provide a differentially expanding balloon 14. For example, distal end 142 may be formed from compliant material and proximal end 144 from non-compliant material to enable a larger distal end 142. Oppositely, proximal end 144 may be formed from compliant material and distal end 142 from non-compliant material to enable a larger proximal end 144. Other means for providing differential expansion of balloon 14 may be used, such as applying energy to different portions of balloon 14 to increase or decrease the compliance, and expandability, of that portion.

Balloon 14 may also be used to direct other equipment into these anatomical locations or be used as an angiographic or hemodynamic monitoring balloon. Differential expansion of balloon 14 may be utilized for proper orientation or direction of such equipment.

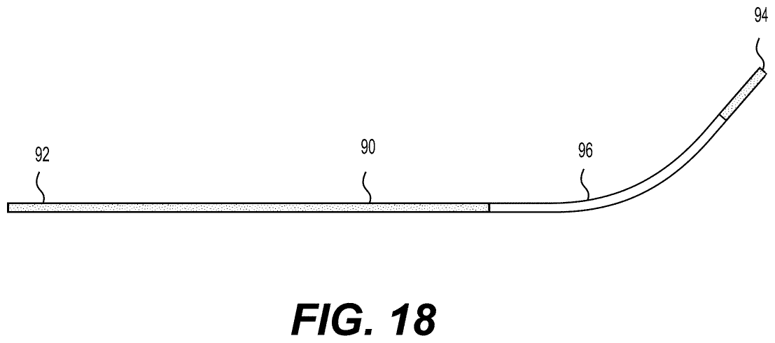

With reference now to FIG. 18, shown is an embodiment of a malleable transseptal needle 90 that may be used with catheter insertion device 10 with a flexible sheath or otherwise capable of multiple angulations. In embodiments, malleable transseptal needle 90 may be of a variety of diameters and lengths. For example, embodiments include an 18 gauge transseptal needle and that is available in 71 cm, 89 cm, and 98 cm lengths. In embodiments, the malleable transseptal needle 90 has different stiffness in a proximal segment 92, distal segment 94, and in a middle segment 96 between. For example, malleable transseptal needle 90 may be stiffer in the proximal segment 92 and distal segment 94 and more flexible (less stiff) in a middle segment or mid-section 96. The mid-section may be the section where catheter insertion device 10 and dilator 16 angulate. In an embodiment, malleable transseptal needle 90 is used and a control handle provided that enables three-dimensional movements. Malleable transseptal needle 90 shown is, preferably, malleable or flexible at least in part. Proximal end 92 of malleable transseptal needle 90 may be stiff (e.g., made from a stiff material, such as a metal). Mid-section or middle 96 of malleable transseptal needle 90 may be malleable or flexible (e.g., made from a flexible, malleable material, such as rubber). Accordingly, mid-section may flex or bend, enabling malleable transseptal needle 90 to pass through angulated or flexed sheath 12.

Distal end 94 of malleable transseptal needle 90 (i.e., end that punctures interatrial cardiac septum) may be stiff with a cap or electrode at its tip for delivering energy to interatrial septum to puncture interatrial septum. In embodiments, transseptal needle is able to transmit radiofrequency energy to create a controlled septal puncture. Such a transseptal needle may or may not be malleable, but is able deliver RF energy through a cap or crown (e.g., an electrode) at its distal end tip. The needle 90 may be connected, e.g., on proximate end (not shown) to a radiofrequency (RF) energy source (not shown) at, e.g., external hub, that provides RF energy through needle to its distal end tip. In such an embodiment, dilator 16 may tent interaxial septum and RF energy capable transseptal needle may create puncture of interaxial septum through delivery of RF energy.

Embodiments may include an additional dilator which would be able to dilate the distal end of sheath 12, or the entire sheath length, thereby significantly increasing the French size of the sheath 12. For example, balloons deployed within sheath 12 may be inflated to expand sheath 12. In such embodiments, catheter insertion device 10 may, therefore, be used to accommodate and deliver larger devices or be able to retrieve devices once they have been extruded from sheath 12 and have embolized. Such balloons may be inflated through one or more hypotubes.

In embodiments, energy, typically electrical energy, may directed through catheter insertion device 10 may be used to increase or decrease the French size of sheath 12. In such embodiments, sheath 12 is fabricated from materials that are known to increase in malleability and or expand when certain energies are applied. In this manner, the French size of sheath 12 may be adjusted to a size deemed necessary during a given procedure. Such energy may be applied through wires or conductive material, connected to energy source external to proximal end of catheter insertion device 10, attached to or fabricated within sheath 12 or other components of catheter insertion device 10. Likewise, parts or portions of catheter insertion device 10 may be selectively made more rigid or more malleable/soft with the application of energy. Therefore, with the application of differential energy to different parts of catheter insertion device 10 at different times, catheter insertion device 10 size may be adjusted to enable various devices that are ordinarily larger and bulkier than the catheter to traverse through the catheter. In embodiments, transseptal insertion device 10 may accommodate devices up to 36 Fr.

In an embodiment of catheter insertion device 10, visualization of an intrathoracic region of interest using MM techniques may be provided. Embodiments may, for example, provide a needle system comprising a hollow needle having a distal portion and a proximal portion, said distal portion having a distal-most end sharpened for penetrating a myocardial wall. The needle may include a first conductor, an insulator/dielectric applied to cover the first conductor over the proximal portion of said needle and a second conductor applied to cover the insulator/dielectric. The method may further direct the needle system into proximity to a myocardial wall, track progress of the needle system using active MRI tracking, penetrate the myocardial wall to approach the intrathoracic region of interest, and, use the needle system as an MRI antenna to receive magnetic resonance signals from the intrathoracic region of interest.

In related embodiments, MRI antenna may be installed on distal tip 13 of sheath 12, dilator 16 or on balloon 14, similar to ultrasound chips or transducers 226 or 326 described above. Wires connecting such MM antenna or other Mill components may pass through lumen in dilator 16 or sheath 12 and connect with appropriate magnetic resonance energy source on exterior of distal end of catheter insertion device 10.

The embodiments of the disclosed invention includes a balloon tipped catheter insertion device which is suitable for facilitating precise and safe cannulation of pulmonary veins. The catheter insertion device includes a sheath that defines at least one lumen therein and has a distal end that is closest to the cardiac structure of a patient when the device is in use and a proximal end that is external to the patient; a balloon that is connected to the distal end of the sheath, wherein the balloon, when inflated and the insertion device is in use, may overhang and extend past the distal end of the sheath, preventing accidental puncturing of the cardiac structures and stabilizing the insertion device against the pulmonary vein; and a dilator that is positioned within the at least one lumen when the insertion device is in use.

The embodiments of the disclosed invention also includes a balloon tipped catheter insertion device which is suitable for facilitating precise and safe cannulation of pulmonary arteries. The catheter insertion device includes a sheath that defines at least one lumen therein and has a distal end that is closest to the cardiac structure of a patient when the device is in use and a proximal end that is external to the patient; a balloon that is connected to the distal end of the sheath, wherein the balloon, when inflated and the insertion device is in use, may overhang and extend past the distal end of the sheath, preventing accidental puncturing of the cardiac structures and stabilizing the insertion device against the pulmonary artery; and a dilator that is positioned within the at least one lumen when the insertion device is in use.

The embodiments of the disclosed invention also includes a balloon tipped catheter insertion device which is suitable for facilitating precise and safe cannulation of pulmonary arteries. The catheter insertion device includes a sheath that defines at least one lumen therein and has a distal end that is closest to the cardiac structure of a patient when the device is in use and a proximal end that is external to the patient; a pigtail catheter is present as well either via a separate lumen or in the main lumen. The pigtail catheter may be a straight catheter with multiple pores for pharmaceutical infusion.

The embodiments of the disclosed invention also includes a balloon tipped catheter insertion device which is suitable for facilitating precise and safe cannulation of pulmonary arteries. The catheter insertion device includes a sheath that defines at least one lumen therein and has a distal end that is closest to the cardiac structure of a patient when the device is in use and a proximal end that is external to the patient; A separate two-way lumen may be present in the main lumen for flushing and aspirating with presence of proximal and distal ports forming a loop of fluid circulation for aspiration either mechanically or using the Venturi effect.

The pulmonary artery and venous balloon tipped catheter insertion devices are either multidirectional or have fixed curls. The pulmonary artery and venous balloon tipped catheter insertion devices may vary from 6 to 42 Fr (French size). The pulmonary artery balloon tipped catheter insertion device may have an aspiration port which could be attached to a mechanical aspiration system or an automated closed loop system capable of applying varying degrees of aspiration force. The pulmonary venous balloon tipped catheter insertion device may have an infusion port for infusing fluids and pharmaceutical agents either mechanically or using an infusion system with varying levels of infusion pressures. The pulmonary artery and venous balloon tipped catheter insertion devices may have balloons which may be used to completely occlude the respective vessels they are introduced into. The pulmonary artery balloon tipped catheter insertion device would also be used to infuse fluids and pharmaceutical agents. The pulmonary artery and venous catheter insertion devices may be connected in a closed loop system whereby the optimal pressure for retrograde perfusion/flushing of the pulmonary vasculature may be performed.

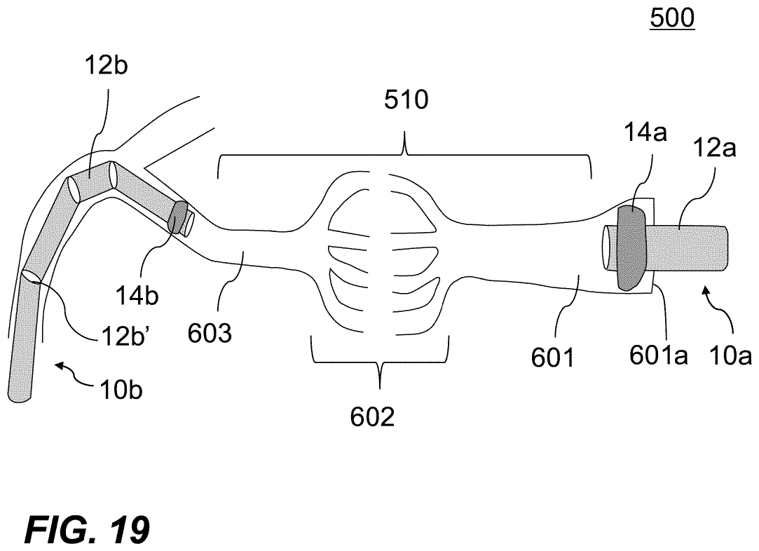

With reference to FIG. 19, shown is closed loop catheter system 500 of the disclosed invention isolating desired segment of pulmonary vascular circulation. The closed loop catheter system 500 includes balloon tipped pulmonary venous catheter insertion device 10a and balloon tipped pulmonary artery catheter insertion device 10b. The pulmonary venous catheter insertion device 10a is configured to be suitable for facilitating precise and safe cannulation of pulmonary veins, and the pulmonary artery catheter insertion device 10b is configured to be suitable for facilitating precise and safe cannulation of pulmonary arteries. The pulmonary venous catheter insertion device 10a and the pulmonary artery catheter insertion device 10b may have a plurality of curls and flexion points for multidirectional deflections. FIG. 19 exemplarily shows the flexion points 12b′ formed on the pulmonary venous catheter insertion device 10a. The flexion points enables the catheter insertions devices 10a, 10b to deflect multi-directionally. The deflection directions may be controlled by the pull wire anchor 40 affixed to distal end of sheath 12 and pull wire actuator 42 as shown in FIG. 14.

When the balloon 14a of the pulmonary venous catheter insertions device 10a is inflated while the catheter insertion devices 10a is in use, the balloon 14a inflated around the distal tip 13a of the catheter insertion device 10a seals ostium 601a of the pulmonary vein 601 and becomes occlusive to flow into and out of the proximal segment of the desired pulmonary vein 601. When the balloon 14b of the pulmonary artery catheter insertions device 10b is inflated while the catheter insertion devices 10b is in use, the distal end of the catheter insertion device 10b is positioned in a desired pulmonary artery and the balloon 14b inflated around the distal tip 13b of the catheter insertion device 10b becomes occlusive to flow into the segmental pulmonary artery. In this configuration, the isolated segment 510 together with the catheter insertion devices 10a, 10b form a closed loop. The isolated segment 510 includes closed segments of the pulmonary vein 601, corresponding pulmonary capillaries 602 and corresponding pulmonary arterial 603.

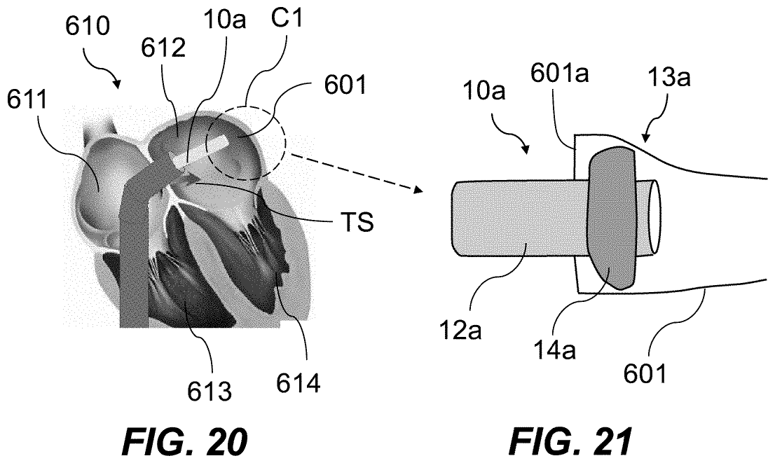

With reference to FIG. 20, shown is balloon tipped pulmonary venous catheter insertion device 10a in the proximal pulmonary vein 601 via trans septal access TS in the heart 610. With reference to FIG. 21, shown is a close view of the portion Cl of FIG. 20 in which balloon tipped pulmonary venous catheter insertion device 10a cannulates and isolates designated pulmonary vein 601 after balloon 14a at distal tip 13a of the venous catheter insertion device 10a is fully inflated. FIG. 20 shows the structure of heart 610 that has right atrium 611, left atrium 612, right ventricle 613 and left ventricle 614. The distal portion of the pulmonary venous catheter insertion device 10a may reach the designated ostium and proximal segment of the designated pulmonary vein 601 via trans septal access TS across the interatrial septum between the right atrium 611 and the left atrium 612. The pulmonary venous catheter insertion device 10a is configured to cannulate the designated pulmonary vein.

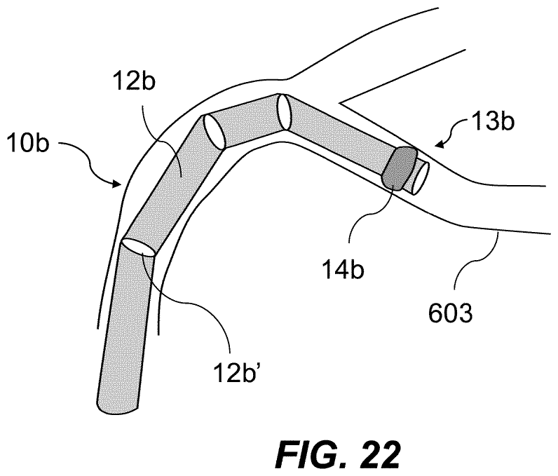

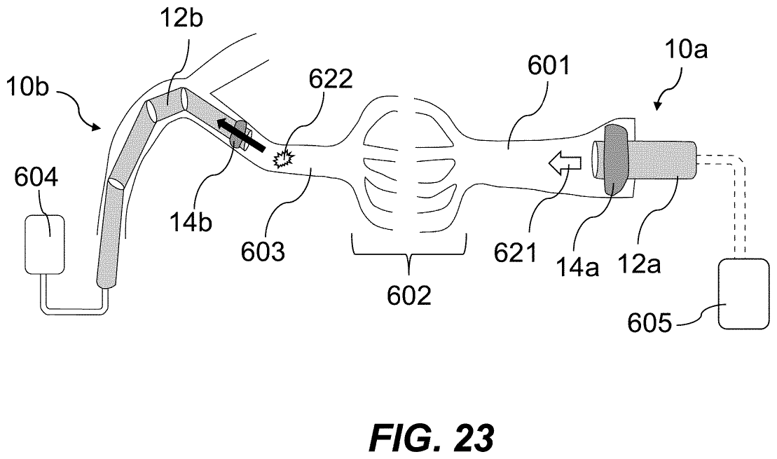

With reference to FIG. 22, shown is balloon tipped pulmonary artery catheter insertion device 10b cannulating and isolating designated pulmonary artery after balloon 14b at distal tip 13b is fully inflated. With reference to FIG. 23, shown is balloon tipped pulmonary artery catheter insertion device 10b cannulating and isolating designated pulmonary artery after distal balloon 14b is inflated and attached to an aspiration apparatus 604 on its proximal end (outside the body) and aspirating matter 622 through the lumen of the balloon tipped pulmonary artery catheter.

Creating A Closed Loop System in the Pulmonary Vasculature.

With reference to FIGS. 19-23, an embodiment of the disclosed invention involves systems and methods for isolating both the desired pulmonary vein and the corresponding pulmonary arterial segment using closed loop catheter system 500 that includes first and second balloon tipped catheter insertion devices 10a and 10b. Once these catheter insertion devices 10a, 10b are cannulated and isolated percutaneously, the cannulated catheter insertion devices 10a, 10b will create an isolated segment 510 which includes closed segments of pulmonary vein 601, corresponding pulmonary capillaries 602, and corresponding pulmonary arterial 603, whereby fluids and/or pharmaceutical agents can be delivered in a retrograde fashion (i.e. from the pulmonary vein into the pulmonary artery using a set rate of flow and designated infusion pressure) into the closed segments of the pulmonary vein 601 then into the pulmonary capillaries 602 and then into the distal and then more proximal corresponding pulmonary artery 603 that is already isolated and cannulated with the pulmonary artery balloon tipped catheter insertion device 10b. The first catheter insertion device 10a includes a sheath 12a and at least one balloon 14a that is connected to the distal end 13a of the sheath 12a.

The second catheter insertion device 10b includes a sheath 12b and at least one balloon 14b that is connected to the distal end 13b of the sheath 12b. This closed loop catheter system 500, which includes the first and second catheter insertion devices 10a and 10b, will serve to create isolated segment 510 as defined by the fact that no material can flow through or around the catheter insertion devices 10a, 10b with inflated balloon tips 14a, 14b into the cannulated vessels (pulmonary artery or pulmonary vein). Any fluid or pharmaceutical introduced into this isolated segment 510 must flow from within the designated catheters. This closed loop catheter system 500 can serve as a closed loop system to (1) dislodge and clear thrombotic material in the distal and proximal pulmonary arterial system that can then be aspirated by the pulmonary arterial balloon tipped catheter or (2) deliver concentrated thrombolytics or other pharmaceuticals using the pulmonary venous balloon tipped catheter into the area affected by thrombus and then removed/aspirated (both thrombotic material and remaining pharmaceutical agent) by the pulmonary artery balloon tipped catheter.

This process occurs with cannulating (via transseptal access) the aforementioned pulmonary venous balloon catheter 10a with the appropriate size based on the pulmonary vein. The distal portion of the pulmonary venous balloon tipped catheter 10a is used to engage the ostium and proximal segment of the desired pulmonary vein 601 using fluoroscopic and echocardiographic guidance. Once the distal portion of the pulmonary venous catheter 10a is in the ostium and proximal segment of the desired pulmonary vein, the balloon 14a at the distal tip 13a of the venous catheter insertion device 10a is inflated to secure its position and create a seal so as not to let anything exit or enter from the ostium of the selected pulmonary vein (see FIG. 21). Next, the balloon tipped catheter insertion device 10b which is suitable for facilitating safe and precise cannulation of the designated and corresponding pulmonary arterial segment is positioned such that the distal tip 13b is within the proximal segment of the desired pulmonary artery 603. Once this is in place, the balloon 14b on the distal tip 13b of the catheter insertion device 10b is inflated to isolate the pulmonary artery 603 meaning that no material can flow around or through it.

Using a Closed Loop System in the Pulmonary Vasculature to Perform Embolectomy.

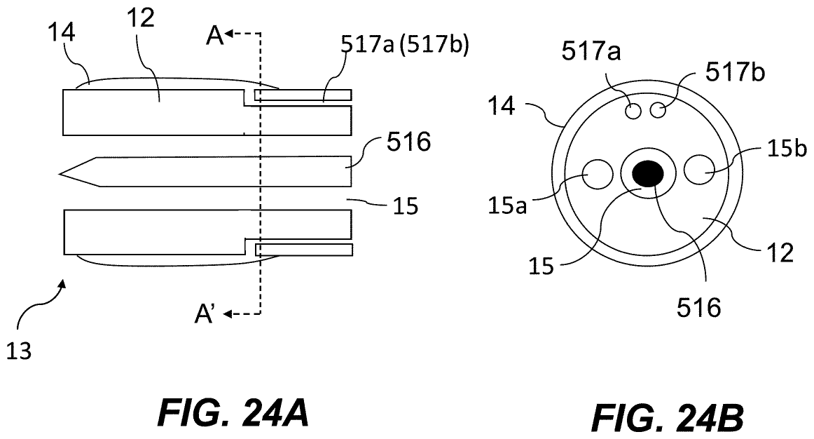

Utilizing the aforementioned closed loop catheter system 500 in the pulmonary vasculature, embolectomy can be performed by infusion of fluid 621 (i.e. saline, heparinized saline, blood, thrombolytics in fluid, any pharmaceutical agent in fluid, or any combination thereof) through the central lumen 15 or another lumens 15a, 15b (see FIGS. 24A-24B) of the pulmonary venous balloon tipped catheter 10a positioned with inflated balloon 14a in the proximal segment of the designated pulmonary vein 601. The infusion will occur at the proximal segment of the pulmonary venous catheter outside the patient's body. The infusion can occur with manual infusion using a large syringe or using a pre-programmed machine that can designate a given volume of fluid at a designated rate of flow and pressure. For this purpose, a proximal end of the pulmonary venous balloon tipped catheter 10a is connected to infuser 605, located outside the patient's body, such as a syringe and a machine that supplies fluid in controlled manners. This infusion will flow into the pulmonary veins 601 and into the pulmonary capillaries 602 and then into the pulmonary arterial vasculature 603. This pressured infusion will then act to dislodge thrombotic material 622 from the pulmonary arterial vascular bed into the more proximal pulmonary artery in question that is already cannulated with a balloon tipped pulmonary arterial catheter 10b with the balloon 14b in an inflated configuration.

Once the infusion is started, then aspiration will be performed through the lumen (such as central lumen 15 or another lumens 15a, 15b) of the balloon tipped pulmonary arterial catheter 10b that is positioned with balloon inflated into the designated pulmonary artery. An aspirator or apparatus 604 for aspiration (i.e. large syringe, mechanical aspiration device) will be attached to the proximal end of the lumen of the balloon tipped catheter 10b in the pulmonary artery. This apparatus 604 will then be used to aspirate fluid and thrombotic material from the isolated pulmonary vasculature, out of the body via the balloon tipped pulmonary artery catheter insertion device 10b (see FIG. 23). Once the procedure is completed, the balloon of the catheter insertion device 10a in the pulmonary vein and the balloon of the catheter insertion device 10b in the pulmonary artery will be deflated and the respective catheter insertion devices will be disengaged from the respective vessels and removed from the body via the same route they were introduced. This technique may also be utilized for the treatment of chronic thromboembolic disease of the pulmonary vasculature in addition to acute pulmonary embolism.

Using a Closed Loop System in the Pulmonary Vasculature to Deliver Pharmaceuticals while Minimizing Systemic Effects of Pharmaceuticals.

Thrombolytics and other pharmaceuticals can be used to treat pulmonary arterial embolism. However, systemic administration in higher concentrations can result in off target effects such as intracranial bleeding. As such, delivering pharmaceuticals into a closed loop system can be used and then the remaining can be aspirated in the closed loop system to prevent said pharmaceutical from making its way into the systemic circulation to avoid off target effects. Utilizing the aforementioned closed loop catheter system 500 in the pulmonary vasculature, pharmaceuticals can be delivered by infusion of said drug in a carrier (i.e. saline, heparinized saline in fluid) through the central lumen 15 of the pulmonary venous balloon tipped catheter 10a positioned with inflated balloon 14a in the proximal segment of the designated pulmonary vein 601. The infusion will occur at the proximal segment of the pulmonary venous catheter outside the patient's body. The infusion can occur with infuser 605 such as manual infusion using a large syringe or using a pre-programmed machine that can designate a given volume of fluid at a designated rate of flow and pressure. This infusion will flow into the pulmonary veins 601 and into the pulmonary capillaries 602 and then into the pulmonary arterial vasculature 603. This pressured infusion will then act in the designated area in the isolated segment of the pulmonary arterial vascular bed and into the more proximal pulmonary artery in question that is already cannulated with a balloon tipped pulmonary arterial catheter 10b with the balloon 14b in an inflated configuration.

Once the infusion is started, then aspiration will be performed through the lumen 15 of the balloon tipped pulmonary arterial catheter 10b that is positioned with balloon 14b inflated into the designated pulmonary artery 603. An aspirator or apparatus 604 for aspiration (i.e. large syringe, mechanical aspiration device) will be attached to the proximal end of the lumen 15 of the balloon tipped catheter 10b in the pulmonary artery. This apparatus will then be used to aspirated fluid, pharmaceutical and thrombotic material 622 from the isolated pulmonary vasculature, out of the body via the balloon tipped pulmonary artery catheter insertion device 10b. Once the procedure is completed, the balloon tipped catheter insertion device 10a in the pulmonary vein and the balloon tipped catheter 10b in the pulmonary artery will be deflated and the respective catheters will be disengaged from the respective vessels and removed from the body via the same route they were introduced.

The closed loop system as described above can be used to filter aspirated blood and reinfuse it into the systemic circulation. The aspirated blood is removed via the pulmonary artery catheter insertion device 10b which can be connected to an apparatus which serves to aspirate blood and other contents from the pulmonary artery into the container whereby it is filtered through and blood products are collected into a reservoir and returned from a reservoir into the circulation using extracorporeal membrane oxygenation (ECMO) system via one of two ways:

a) Venous system: Blood is removed from the reservoir using an ECMO system which then diverts blood (with use of a pump) into a cannula placed into the central venous system either via common femoral vein or internal jugular vein access.

b) Arterial system: Blood is removed from the reservoir using an ECMO system which then diverts blood (with use of a pump) into an oxygenator which is then diverted via tubing into a cannula in the femoral artery.

With reference to FIG. 24A-24B, shown are a side view of the distal tip 13 portion and a cross-sectional view of the section A-A′ of the distal tip 13 portion of the balloon tipped catheter insertion devices 10a, 10b which are used for the closed loop catheter system 500 of the disclosed invention. In the embodiments, the balloon 14 on the sheath 12 may have separate hypotubes 517a, 517b for inflation and deflation. For example, the sheath 12 may have inflation hypotube 517a and a deflation hypotube 517b to inflate and deflate the balloon 14, respectively. However, the embodiment is not limited to this configuration. The balloons 14 may have a hypotube that may be used for both inflation and deflation. The balloon 14 may be inflated and deflated through the same hypotube. The balloon 14 of pulmonary artery and venous balloon tipped catheter insertion device 10 may be used to completely occlude the respective vessels into which they are introduced. The catheter insertion device 10 has a center lumen 15 in which a wire member or dilator 516 is movably disposed. The dilator 516 may be a wire, Brockenbrough needle, radiofrequency tip needle, pigtail catheter, or transseptal wire.

In an embodiment, the catheter insertion device 10 may have additional lumens 15a, 15b for flushing and aspirating with presence of proximal and distal ports forming a loop of fluid circulation for aspiration either mechanically or using the Venturi effect. However, the disclosed invention is not limited to this configuration. The catheter insertion device 10 may be configured to have one lumen 15 for dilator and flushing and/or aspirating, or may have more than one lumen for dilator, flushing and aspirating. When the catheter insertion device 10 has a multiple lumens, one lumen 15a, for example, may be used for an aspiration port which may be attached to a mechanical aspiration system or an automated closed loop system capable of applying varying degrees of aspiration force, and the other lumen 15b may be used for an infusion port for infusing fluids and pharmaceutical agents either mechanically or using an infusion system with varying levels of infusion pressures. The pulmonary artery balloon tipped catheter insertion device 10 may be used to infuse fluids and pharmaceutical agents through the one or more lumens. The pulmonary artery and venous catheter insertion devices 10a, 10b may be connected in a closed loop system whereby the optimal pressure for retrograde perfusion/flushing of the pulmonary vasculature may be performed.

In an embodiment, various types of wire members or dilators 516 may be separately disposed on one or more lumens. For example, one of the wire members 516 may be a pigtail catheter, and the pigtail catheter may be present as well either via a separate lumen 15a or 15b or in the center lumen 15. The pigtail catheter may be a straight catheter with multiple pores for pharmaceutical infusion.

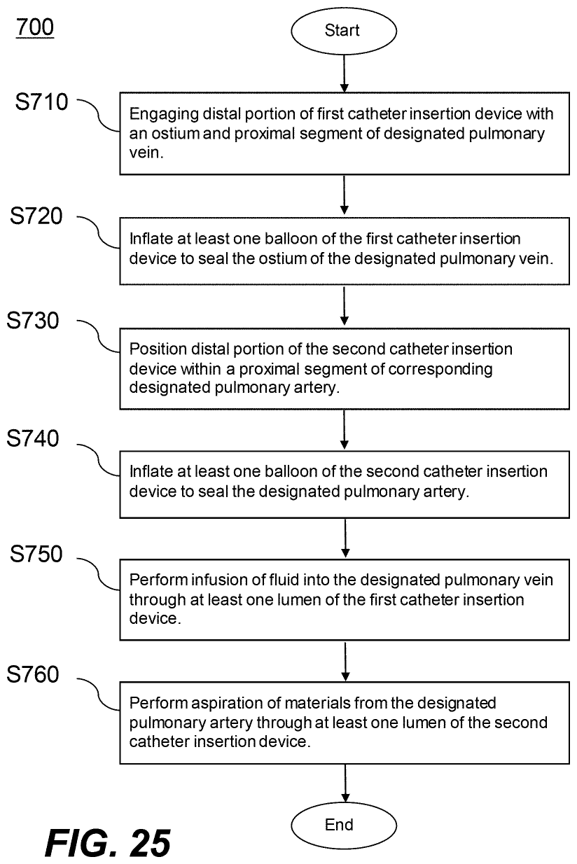

With reference to FIG. 25, shown is a flowchart diagram for a method of using the closed loop catheter system 500 that includes a first balloon tipped catheter insertion device 10a and a second balloon tipped catheter insertion device 10b for pulmonary embolism. The method 700 includes steps of engaging a distal portion of a first catheter insertion device with an ostium and proximal segment of a designated pulmonary vein, block S710, inflating at least one balloon of the first catheter insertion device to seal the ostium of the designated pulmonary vein, block S720, positioning a distal portion of the second catheter insertion device is within a proximal segment of a corresponding designated pulmonary artery, block S730, inflating at least one balloon of the second catheter insertion device to seal the designated pulmonary artery, block S740, performing infusion of fluid into the designated pulmonary vein through at least one lumen defined in a sheath of the first catheter insertion device by using an infuser connected to the first catheter insertion device, block S750, and performing aspiration of materials from the designated pulmonary artery through at least one lumen defined in a sheath of the second catheter insertion device by using an aspirator connected to the second catheter insertion device, block S760.

Since many modifications, variations, and changes in detail can be made to the described preferred embodiments of the invention, it is intended that all matters in the foregoing description and shown in the accompanying drawings be interpreted as illustrative and not in a limiting sense. Consequently, the scope of the invention should be determined by the appended claims and their legal equivalents.

What is claimed is:

1. A catheter system for pulmonary embolism with balloon tipped catheter insertion devices, comprising:
a first catheter insertion device configured to be suitable for facilitating precise and safe cannulation of pulmonary veins, wherein the first catheter insertion device is configured to be connected to an infuser to supply fluid to the pulmonary veins, the first catheter insertion device comprising:
a sheath that defines at least one lumen therein and has a distal end that is configured to be positioned at proximal segment of a designated pulmonary vein and a proximal end that is external to the patient; and
at least one balloon that is positioned at the distal end of the sheath, wherein the at least one balloon is configured to seal ostium of the designated pulmonary vein when inflated; and
a second catheter insertion device configured to be suitable for facilitating precise and safe cannulation of pulmonary arteries, wherein the second catheter insertion device is configured to be connected to an aspirator to aspirate materials from the pulmonary arteries, the second catheter insertion device comprising:
a sheath that defines at least one lumen therein and has a distal end that is configured to be positioned at proximal segment of a designated pulmonary artery and a proximal end that is external to the patient; and
at least one balloon that is positioned at the distal end of the sheath, wherein the at least one balloon is configured to seal the designated pulmonary artery when inflated.

2. The catheter system of claim 1 wherein the fluid includes one or more selected from a group consisting of saline, heparinized saline, blood, thrombolytics in fluid, and pharmaceutical agent in fluid.

3. The catheter system of claim 1 wherein the at least one lumen of the first catheter insertion device is configured to deliver the fluid supplied by the infuser.

4. The catheter system of claim 1 wherein the first catheter insertion device comprises a dilator movably positioned in the at least one lumen, wherein the dilator is configured to puncture septum.

5. The catheter system of claim 4 wherein the dilator includes a pigtail catheter having multiple pores for pharmaceutical infusion.

6. The catheter system of claim 1 wherein the sheath of the first catheter insertion device defines one or more additional lumens to deliver additional fluids into the designated pulmonary vein.

7. The catheter system of claim 1 wherein the first catheter insertion device has a plurality of curls and flexion points for multidirectional deflections.

8. The catheter system of claim 1 wherein the at least one lumen of the second catheter insertion device is configured to carry the materials to the aspirator.

9. The catheter system of claim 1 wherein the second catheter insertion device comprises a dilator movably positioned in the at least one lumen, wherein the dilator is configured to puncture septum.

10. The catheter system of claim 1 wherein the sheath of the second catheter insertion device defines one or more additional lumens for flushing or aspirating.

11. The catheter system of claim 1 wherein the second catheter insertion device has a plurality of curls and flexion points for multidirectional deflections.

12. A method of using a catheter system including a first catheter insertion device and a second catheter insertion device for pulmonary embolism, comprising:
engaging a distal portion of a first catheter insertion device with an ostium and proximal segment of a designated pulmonary vein;
inflating at least one balloon of the first catheter insertion device to seal the ostium of the designated pulmonary vein;
positioning a distal portion of the second catheter insertion device within a proximal segment of a corresponding designated pulmonary artery;
inflating at least one balloon of the second catheter insertion device to seal the designated pulmonary artery;
performing infusion of fluid into the designated pulmonary vein through at least one lumen defined in a sheath of the first catheter insertion device by using an infuser connected to the first catheter insertion device; and
performing aspiration of materials from the designated pulmonary artery through at least one lumen defined in a sheath of the second catheter insertion device by using an aspirator connected to the second catheter insertion device.

13. The method of claim 12 wherein the fluid includes one or more selected from a group consisting of saline, heparinized saline, blood, thrombolytics in fluid, and pharmaceutical agent in fluid.

14. The method of claim 12 wherein the engaging the distal portion of the first catheter insertion device is performed by using a fluoroscopic or echocardiographic guidance.

15. The method of claim 12 wherein the infusion of fluid is performed such that the fluid flows into the designated pulmonary vein, corresponding pulmonary capillaries and the corresponding designated pulmonary artery.

16. The method of claim 12 wherein the infusion of fluid is performed with a designated rate of flow and pressure to dislodge thrombotic materials from a pulmonary arterial vascular bed into the proximal segment of the corresponding designated pulmonary artery.

17. The method of claim 12 wherein the performing aspiration of materials includes removing thrombotic materials from the designated pulmonary artery.

18. The method of claim 12 wherein the performing infusion of fluid comprises infusing pharmaceuticals into the designated pulmonary vein, and the performing aspiration of materials comprises aspirating the pharmaceuticals and thrombotic materials through the designated pulmonary artery.

19. The method of claim 12 wherein the performing aspiration of materials includes aspirating blood from the designated pulmonary artery, and the method further comprises:
filtering the blood; and
reinfusing the filtered blood into circulations using extracorporeal membrane oxygenation (ECMO) system.
